(19) United States
(12) Patent Application Publication
(10) Pub. No.: US 2010/0063343 A1
(43) Pub. Date: Mar. 11, 2010

(54) PROCESS FOR THE PURIFICATION OF METHANE GAS

(76) Inventors: Guy D. Cusumano, Holmdel, NJ (US); Clint C. Harriman, Avon, CT (US)

Correspondence Address: Guy Cusumano, 12 Winding Brook Way, Holmdel, NJ, 07733, US

(21) Appl. No.: 12/557,671

(22) Filed: Sep. 11, 2009

Related U.S. Application Data
(60) Provisional application No. 61/095,971, filed on Sep. 11, 2008.

(51) Int. Cl. C07C 7/00

(52) U.S. Cl. 585/800

(57) ABSTRACT

Process for purification of landfill gas and digester gas, also known as biogas or biomethane, to remove harmful constituents within the stream. The harmful constituents treated include water, particulate, sulfur (hydrogen sulfide), and siloxanes.

This application claims priority to provisional application No. 61/095,971 filed Sep. 11, 2008.

### BACKGROUND OF THE INVENTION AND RELATED ART

This invention generally relates to the cleaning and recovery of a methane fuel from landfill gas, and digester gas, or other methane sources. More particularly, the invention provides an improved process for concentrating and removing certain commonly occurring pollutants from landfill gas, and digester gas using an equipment set employing proven individual technologies here combined, balanced, and working together in the way designed by the inventors, to produce an economical, safe fuel product. We call this process Process 10.

The cleaned methane fuel may be pressurized into a high pressure fuel which is suitable for use with motors or vehicle engines adapted to be fueled by compressed natural gas. The landfill gas is generated by the decomposition of buried waste or garbage. Digester gas is generated by the anaerobic digestion of wastewater in a sewage treatment plant. Both gases are principally comprised of methane and carbon dioxide together with small amounts of other constituents which may include nitrogen, oxygen, hydrogen, carbon monoxide and a variety of trace contaminants. Boilers, internal combustion engines, and turbine engines experience operating difficulties, and failures when exposed to some of these constituents. The Process 10 equipment is designed to remove these detrimental components rendering the resulting fuel product safe for such engines.

Because of their high methane content, landfill gas, and digester gas have attracted much attention as potential fuel sources. However, in order to utilize them as a substitute for natural gas in existing fuel distribution systems or as a fuel for internal combustion engines, it is necessary to remove the detrimental constituents, primarily Hydrogen sulfide, and siloxanes, as well as moisture, which is a problem when it reacts with the hydrogen sulfide.

There are several technically workable, relatively expensive methane purification processes available for the purification of landfill gas, and digester gas. They produce either a pipeline quality methane product, or a near pipeline quality methane product. The subject of this invention, Process 10, cleans away the primary contaminants in landfill gas and digester gas to render the product gas better for fuel use. Many engines can run on this medium BTU fuel, if the fuel gas has been cleaned adequately, which is what the Process 10 process is designed to accomplish. By selecting the main engine damaging contaminants, and removing them, with the lowest cost effective procedures, in a single arrangement of equipment, balanced, and optimally organized, many more landfill gas and digester gas sources can be economically developed as useful energy projects using Process 10.

RELATED U.S. PATENT DOCUMENTS

| Application # | Filing Date | Patent # | Issue Date |
|---|---|---|---|
| 08/462,692 | Jun. 5, 1995 | 5,730,784 | Mar. 24, 1998 |
| 11/092,684 | Mar. 30, 2005 | 7,306,652 | Dec. 11, 2007 |

### CURRENT US CLASS: 95, 96, 60, 62

### CURRENT INTERNATIONAL CLASS: 53/04

### FIELD OF SEARCH: 95/96

U.S. Pat. No. 5,730,784, discloses a sulfur separation process for the purification of the gas stream from sulfur (H2S).

U.S. Pat. No. 7,306,652, discloses a siloxane separation process for the purification of the gas stream from siloxanes.

### SUMMARY OF THE INVENTION

### Purify Methane Gases

The energy contained in Landfill Gas, and Digester Gas is directly proportional to the methane, and other trace hydrocarbons they contain. Both gases contain roughly 40 to 60% methane, and carbon dioxide. This invention enables the cleaning and reduction of certain constituents of Landfill Gas and Digester Gas resulting in a medium BTU fuel that can be used with a minimum of unfavorable side effects in many applications including heating, electric generation in a genset, and some vehicle engines with only minor tuning modifications.

The ability to derive useful work from the energy in either Landfill Gas (LFG), or Digester Gas mixtures requires cleaning from the gas mixtures the contaminants that are the most damaging to mechanical equipment. All fuel and engine usage causes mechanical wear over time. When all of the typical constituents of Landfill Gas and Digester Gas are present, this wear can be excessive and, along with other issues related to operating with dirty biogas, cause premature equipment failures. By identifying, and substantially cleaning the gas mixtures of a high percentage of these harmful constituents that lead to premature failure, more nearly normal mechanical useful lives can be obtained from equipment using the fuel thus cleaned.

The gas cleaning methodology of this invention is designed to be low cost, to enable the maximum number of Landfill Gas and Digester Gas sources to be converted to useful applications. By only substantially removing the offending constituents that lead to premature equipment wear, the total cost of Landfill Gas and Digester Gas fuel preparation is kept to a minimum.

The four constituents substantially removed from Landfill Gas and Digester Gas by this invention are water, particulate matter, hydrogen sulfide and siloxanes. The order of removal is of primary importance to both protect the removal equipment, and optimize the efficiency, and therefore net cost of both the removal equipment, and the operating cost of the removal equipment. A set of self contained skids can contain all of the equipment, fully assembled, and calibrated to clean each project Landfill Gas, or Digester Gas stream.

The invention first accepts the Methane Gas through a primary knockout pot that removes droplets, and filters matter from the Methane Gas. Main vacuum and/or blower's pressure boost the gas to the appropriate conditions for moving the process gases as required. Hydrogen Sulfide Removal. The hydrogen sulfide (H2S) removal system shall either be a scrubber with solid media that absorbs the H2S in the Methane Gas stream or a liquid scrubber that catalytically converts H2S in the gas stream to solid sulfur. When using the second mentioned method, hydrogen sulfide removal produces inert element sulfur that can be land filled in contrast to typical hydrogen sulfide removal processes that produce a hazardous waste element to be disposed of as hazardous waste (first mentioned method).

The gas after the hydrogen sulfide removal process moves to the siloxane removal equipment where siloxanes are removed by adsorption. These siloxane waste products are then disposed of with a small amount of Methane Gas in a flare or thermal oxidizer. The resulting cleaned Methane Gas fuel is delivered as feedstock to the work application with clean, dry, filtered and temperature/dew point controlled fuel gas stream, without excessive hydrogen sulfide, and siloxanes.

### BRIEF DESCRIPTION OF THE DRAWINGS

FIG. 1 is a flow diagram for the Standard arrangement for Methane Gas purification treatment system removing sulfur and siloxanes.

FIG. 2 is a flow diagram for an Alternate arrangement of prime mover for Methane Gas purification treatment system removing sulfur and siloxanes.

FIG. 3 is a flow diagram for an Alternate arrangement of prime mover for Methane Gas purification treatment system removing sulfur and siloxanes.

### DETAILED DESCRIPTION OF THE DRAWINGS

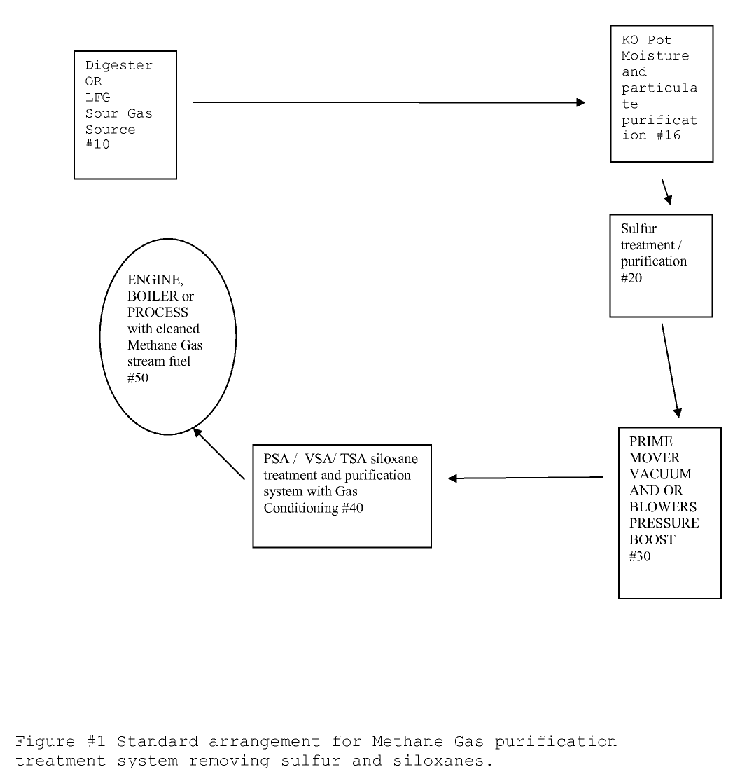

### Description for FIG. 1 Standard Arrangement for Methane Gas Purification Treatment System Removing Sulfur and Siloxanes

### Block # Purpose Discussion

10: Source Methane Gas from many sources, for example digesters, landfills, sour gas wells, and the like. The invention is directed to gas purification processes, and more specifically, to a process for purification and cleaning of Methane Gases containing moisture, particulate, sulfur and siloxanes.
16: Knock Out Pot Moisture and Particulate filtration: Methane Gas taken from 10 is pre treated at the inlet point for the process. This step specifically is for the cleaning of Methane Gases moisture droplets and particulates.
20: Sulfur treatment stage: Pre treated Methane Gas is treated to remove sulfur. Hydrogen Sulfide Removal. The hydrogen sulfide (H2S) removal system shall either be a scrubber with solid media that absorbs H2S in the Methane Gas stream or a liquid scrubber that catalytically converts H2S in the gas stream to solid sulfur.
30: Prime mover: Differential pressure blower, compressor, vacuum pump stage. This stage produces the required delta P to move the gas within the process stages, deliver it at pressures required for use in the engine/boiler user system. This stage moves from point to point within the flow path per job requirements.
40: Siloxanes Removal. The siloxanes removal system shall be granular media scrubbers with regenerable or non-regenerable adsorbent media or a pressure swing adsorption (PSA) system, Vacuum Swing Adsorption system (VSA), Temperature Swing Adsorption (TSA) system, with a final particulate filter to remove any entrained particulates in the gas stream. This stage may include Gas Conditioning. A skid mounted gas drying system, including a moisture inlet coalescer, heat exchangers, chiller, pumps, moisture separator, recirculation bypass, and all piping, controls, and control panel.
50: Prime user: engine/turbine/boiler end user gas system.

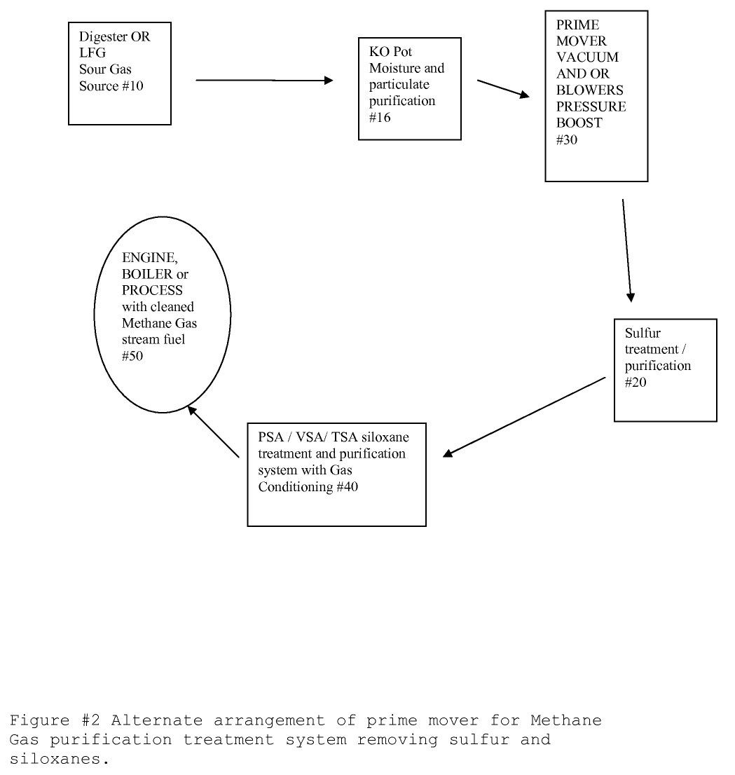

### Description for FIG. 2 Standard Arrangement for Methane Gas Purification Treatment System Removing Sulfur and Siloxanes.

### Block # Purpose Discussion

10: Source Methane Gas from many sources, for example digesters, landfills, sour gas wells, and the like. The invention is directed to gas purification processes, and more specifically, to a process for purification and cleaning of Methane Gases containing moisture, particulate, sulfur and siloxanes.
16: Knock Out Pot Moisture and Particulate filtration: Methane Gas taken from 10 is pre treated at the inlet point for the process. This step specifically is for the cleaning of Methane Gases moisture droplets and particulates.
30: Prime mover: Differential pressure blower, compressor, vacuum pump stage. This stage produces the required delta P to move the gas within the process stages, deliver it at pressures required for use in the engine/boiler user system. This stage moves from point to point within the flow path per job requirements.
20: Sulfur treatment stage: Pre treated Methane Gas is treated to remove sulfur. Hydrogen Sulfide Removal. The hydrogen sulfide (H2S) removal system shall either be a scrubber with solid media that absorbs H2S in the Methane Gas stream or a liquid scrubber that catalytically converts H2S in the gas stream to solid sulfur.
40: Siloxanes Removal. The siloxanes removal system shall be granular media scrubbers with regenerable or non-regenerable adsorbent media or a pressure swing adsorption (PSA) system, Vacuum Swing Adsorption system (VSA), Temperature Swing Adsorption (TSA) system, with a final particulate filter to remove any entrained particulates in the gas stream. This stage may include Gas Conditioning. A skid mounted gas drying system, including a moisture inlet coalescer, heat exchangers, chiller, pumps, moisture separator, recirculation bypass, and all piping, controls, and control panel.
50: Prime user: engine/turbine/boiler end user gas system.

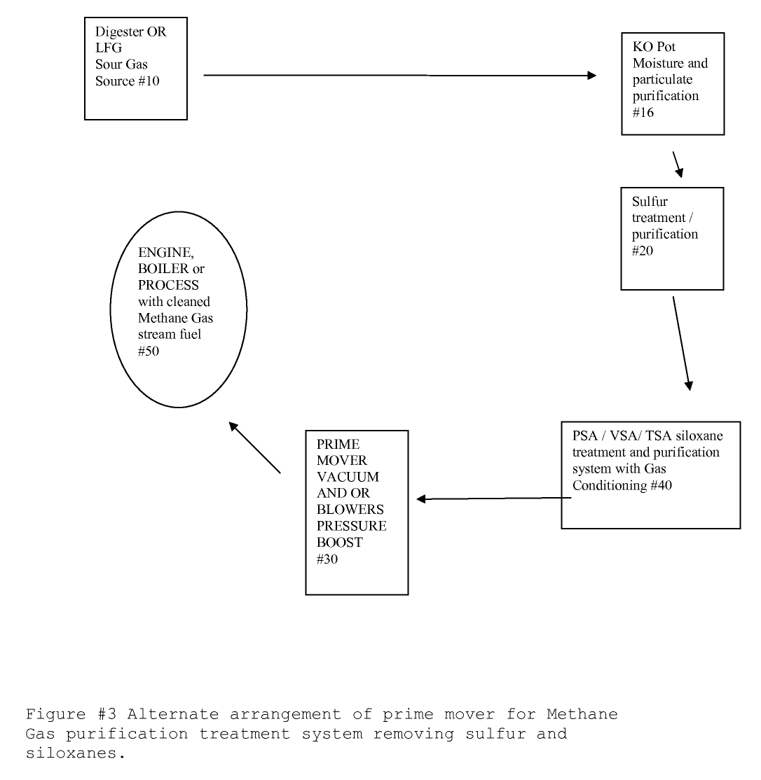

### Description for FIG. 3 Standard Arrangement for Methane Gas Purification Treatment System Removing Sulfur and Siloxanes.

### Block # Purpose Discussion

10: Source Methane Gas from many sources, for example digesters, landfills, sour gas wells, and the like. The invention is directed to gas purification processes, and more specifically, to a process for purification and cleaning of Methane Gases containing moisture, particulate, sulfur and siloxanes.
16: Knock Out Pot Moisture and Particulate filtration: Methane Gas taken from 10 is pre treated at the inlet point for the process. This step specifically is for the cleaning of Methane Gases moisture droplets and particulates.
20: Sulfur treatment stage: Pre treated Methane Gas is treated to remove sulfur. Hydrogen Sulfide Removal. The hydrogen sulfide (H2S) removal system shall either be a scrubber with solid media that absorbs H2S in the Methane Gas stream or a liquid scrubber that catalytically converts H2S in the gas stream to solid sulfur.
40: Siloxanes Removal. The siloxanes removal system shall be granular media scrubbers with regenerable or non-regenerable adsorbent media or a pressure swing adsorption (PSA) system, Vacuum Swing Adsorption system (VSA), Temperature Swing Adsorption (TSA) system, with a final particulate filter to remove any entrained particulates in the gas stream. This stage may include Gas Conditioning. A skid mounted gas drying system, including a moisture inlet coalescer, heat exchangers, chiller, pumps, moisture separator, recirculation bypass, and all piping, controls, and control panel.
30: Prime mover: Differential pressure blower, compressor, vacuum pump stage. This stage produces the required delta P to move the gas within the process stages, deliver it at pressures required for use in the engine/boiler user system. This stage moves from point to point within the flow path per job requirements.
50: Prime user: engine/turbine/boiler end user gas system.

### SUMMARY OF DISCLOSURE

In summary of this disclosure, the present invention provides novel method and apparatus for effecting the removal of components from gas streams, such as by chemical reactions or physical separation. Modifications are possible within the scope of this invention.

What is claimed is:

1. A gas purifying process for cleaning of contaminants harmful to engines, boilers, and other gas burning apparatus, specifically moisture, particulate matter, sulfur and siloxanes from landfill gas, digester gas, and other methane sources.
